(19) United States
(12) Patent Application Publication
(10) Pub. No.: US 2025/0248927 A1
(43) Pub. Date: Aug. 7, 2025

(54) Implantable Device for Release of Gonadotropin-Releasing Hormone Agonist or Antagonist

(71) Applicant: Celanese EVA Performance Polymers LLC, Irving, TX (US)

(72) Inventors: Jeffrey Haley, Norwood, OH (US); Minakshi Bhardwaj, Independence, KY (US); Harsh Patel, Union, KY (US)

(21) Appl. No.: 19/036,097

(22) Filed: Jan. 24, 2025

Related U.S. Application Data
(60) Provisional application No. 63/627,828, filed on Feb. 1, 2024.
(60) Provisional application No. 63/693,732, filed on Sep. 12, 2024.

(51) Int. Cl. A61K 9/00; A61K 38/09; A61K 47/32

(52) U.S. Cl. CPC A61K 9/0024; A61K 38/09; A61K 47/32

(57) ABSTRACT

An implantable device for delivery of a therapeutic agent is provided. The device includes a core including a core polymer matrix within which is dispersed a therapeutic agent. The therapeutic agent includes one or more GnRH agonist or antagonists or antagonists. The core polymer matrix includes an ethylene vinyl acetate copolymer having a melting temperature of from about 20° C. to about 100° C. as determined in accordance with ASTM D3418-15 and/or a melt flow index of from about 0.2 to about 100 grams per 10 minutes as determined in accordance with ASTM D1238-13 at a temperature of 190° C. and a load of 2.16 kilograms.

### RELATED APPLICATIONS

The present application is based upon and claims priority to U.S. Provisional Patent Application Ser. No. 63/627,828, having a filing date of Feb. 1, 2024, and U.S. Provisional Patent Application Ser. No. 63/693,732, having a filing date of Sep. 12, 2024, which are incorporated herein by reference.

### BACKGROUND OF THE INVENTION

Gonadotropin-releasing Hormone (GnRH) agonists and antagonists have been used for treating breast cancer, ovarian cancer, prostate cancer, reproductive disorders, uterine leiomyoma, and endometriosis. GnRH agonist or antagonists are also known as luteinizing-hormone-releasing hormone (LHRH) agonists and antagonists and can be used to block the secretion of endogenous gonadotropins. Treatment with GnRH agonist or antagonists can be long lasting and can require treatment for many weeks, months, or even years. Patients taking oral dosage forms may be required to take the oral dose at least one per day, if not multiple times per day, for the duration of treatment. Patients taking injectable dosage forms may have to administer multiple injections each month. Such continuous dosage over a long period of time can result in decrease patient compliance, as many patients either forget to take the medication or become tired of having to continually take the medication.

As such, a need continues to exist for treatment option that are capable of delivering one or more GnRH agonist or antagonists over a sustained period of time.

### SUMMARY OF THE INVENTION

In accordance with one embodiment of the present invention, an implantable device for delivery of a therapeutic agent including a GnRH receptor agonist is disclosed. The device comprises a core comprising a core polymer matrix within which is dispersed a therapeutic agent including one or more GnRH agonist or antagonists. The core polymer matrix includes an ethylene vinyl acetate copolymer having a melting temperature of from about 20° C. to about 100° C. as determined in accordance with ASTM D3418-15 and/or a melt flow index of from about 0.2 to about 100 grams per 10 minutes as determined in accordance with ASTM D1238-13 at a temperature of 190° C. and a load of 2.16 kilograms.

Other features and aspects of the present invention are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended drawings in which:

FIG. 1 is a perspective view of one embodiment of the implantable device of the present invention;

FIG. 2 is a cross-sectional view of the implantable device of FIG. 1;

FIG. 3 is a perspective view of another embodiment of the implantable device of the present invention;

FIG. 4 is a cross-sectional view of the implantable device of FIG. 3;

FIG. 5 illustrates the cumulative release (mg) per day of goserelin acetate for Examples 1-3;

FIG. 6 illustrates HPLC chromatograms taken at Day 1 for Examples 1-3; and

FIG. 7 illustrates HPCL chromatograms taken at Day 92 for Examples 1-3.

Repeat use of references characters in the present specification and drawing is intended to represent same or analogous features or elements of the invention.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to an implantable device that is capable of delivering a GnRH agonist or antagonist for prohibiting and/or treating a condition, disease, and/or cosmetic state in a patient (e.g., human, pet, farm animal, racehorse, etc.). The condition and/or disease can include cancer, such as prostate or breast cancer. The implantable device includes a core comprising a core polymer matrix within which is dispersed a therapeutic agent including one or more GnRH agonist or antagonists. The core polymer matrix includes an ethylene vinyl acetate copolymer and has a melting temperature of from about 20° C. to about 100° C. as determined in accordance with ASTM D3418-15 and a melt flow index of from about 0.2 to about 100 grams per 10 minutes as determined in accordance with ASTM D1238-13 at a temperature of 190° C. and a load of 2.16 kilograms.

Various embodiments of the present invention will now be described in more detail.

### I. Core

As indicated above, the core polymer matrix contains at least a polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the core polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-13 at a temperature of 190° C. and a load of 2.16 kilograms.

In certain embodiments, the core polymer matrix may contain a semi-crystalline olefin copolymer. The melting temperature of such an olefin copolymer may, for instance, range from about 40° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt. % to about 60 wt. %, in some embodiments about 20 wt. % to about 60 wt. %, and in some embodiments, from about 25 wt. % to about 50 wt. %. Conversely, the olefin monomeric content of the copolymer may be likewise within a range of from about 40 wt. % to about 90 wt. %, in some embodiments about 40 wt. % to about 80 wt. %, and in some embodiments, from about 50 wt. % to about 75 wt. %.

In one particular embodiment, for example, the core polymer matrix may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA®4030AC); Dow under the designation ELVAX® (e.g., ELVAX®40W); and Arkema under the designation EVATANE® (e.g., EVATANE 40-55). In embodiments, the ethylene vinyl acetate copolymer in the core polymer matrix is from about 20 wt. % to about 90 wt. %, such as from about 30 wt. % to about 80 wt. %, such as from about 40 wt. % to about 70 wt. %.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and polymer matrix have a melting temperature and/or melt flow index within the range noted above. For example, the polymer matrix may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second copolymer may likewise constitute from about 20 wt. % to about 80 wt. %, in some embodiments from about 30 wt. % to about 70 wt. %, and in some embodiments, from about 40 wt. % to about 60 wt. % of the polymer matrix.

In certain cases, ethylene vinyl acetate copolymer(s) constitute the entire polymer content of the core polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

One or more therapeutic agents (e.g., GnRH agonist or antagonists) are also dispersed within the core polymer matrix that are capable of prohibiting and/or treating a condition, disease, and/or cosmetic state a patient. The therapeutic agent may be prophylactically, therapeutically, and/or cosmetically active, systemically, or locally. The therapeutic agent can be homogenously dispersed within the core polymer matrix. Typically, therapeutic agents will constitute from about 5 wt. % to about 60 wt. %, in some embodiments from about 10 wt. % to about 50 wt. %, and in some embodiments, from about 15 wt. % to about 45 wt. % of the core, while the core polymer matrix constitutes from about 40 wt. % to about 95 wt. %, in some embodiments from about 50 wt. % to about 90 wt. %, and in some embodiments, from about 55 wt. % to about 85 wt. % of the core. In certain embodiments, therapeutic agents will constitute from about 20 wt. % to about 80 wt. % of the core, and in some embodiments, from about 30 wt. % to about 70 wt. % of the core.

The therapeutic agent can be either naturally occurring or man-made by any method known in the art. Typically, it is also desired that the therapeutic agent is stable at high temperatures so that it can be incorporated into the polymer matrix at or near the melting temperature of the hydrophobic polymer employed in the core polymer matrix. For example, the therapeutic agent typically remains stable at temperatures of from about 25° C. to about 120° C., in some embodiments from about 40° C. to about 110° C., in some embodiments from about 40° C. to about 100° C., in some embodiments from about 40° C. to about 80° C., and in some embodiments, from about 50° C. to about 70° C. Suitable therapeutic agents will be further discussed hereinbelow.

The core may also optionally contain one or more excipients if so desired, such as radiocontrast agents, release modifiers, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the core. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc.

To help further control the release rate from the implantable device, a hydrophilic compound may also be incorporated into the core that is soluble and/or swellable in water. When employed, the weight ratio of the ethylene vinyl acetate copolymer(s) the hydrophilic compounds within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the core can include a plurality of water-soluble particles distributed within the core polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of the core.

Regardless of the particular components employed, the core may be formed through a variety of known techniques, such as by hot-melt extrusion, injection molding, solvent casting, dip coating, spray coating, microextrusion, coacervation, compression molding (e.g., vacuum compression molding), etc. In one embodiment, a hot-melt extrusion technique may be employed. Hot-melt extrusion is generally a solvent-free process in which the components of the core (e.g., hydrophobic polymer, therapeutic agent(s), optional excipients, etc.) may be melt blended and optionally shaped in a continuous manufacturing process to enable consistent output quality at high throughput rates. This technique is particularly well suited to various types of hydrophobic polymers, such as olefin copolymers. Namely, such copolymers typically exhibit a relatively high degree of long-chain branching with a broad molecular weight distribution. This combination of traits can lead to shear thinning of the copolymer during the extrusion process, which help facilitates hot-melt extrusion. Furthermore, the polar comonomer units (e.g., vinyl acetate) can serve as an “internal” plasticizer by inhibiting crystallization of the polyethylene chain segments. This may lead to a lower melting point of the olefin copolymer, which improves the overall flexibility of the resulting material and enhances its ability to be formed into devices of a wide variety of shapes and sizes.

During a hot-melt extrusion process, melt blending may occur at a temperature range of from about 20° C. to about 200° C., in some embodiments, from about 30° C. to about 150° C., in some embodiments from about 40° C. to about 100° C., and in some embodiments, in some embodiments from about 100° C. to about 120° C., to form a polymer composition. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or therapeutic agent(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be in the form of pellets, sheets, fibers, filaments, etc., which may be shaped into the core using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the core. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape of the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 30° C. to about 120° C., in some embodiments from about 60° C. to about 110° C., and in some embodiments, from about 30° C. to about 60° C.

As indicated above, another suitable technique for forming a core of the desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present disclosure. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

### II. Therapeutic Agent

One or more therapeutic agents are also dispersed within the core polymer matrix that are capable of prohibiting and/or treating a condition, disease, and/or cosmetic state a patient. The therapeutic agent may be prophylactically, therapeutically, and/or cosmetically active, systemically or locally. The therapeutic agent can include one or more GnRH agonist or antagonists or antagonists. GnRH agonist or antagonists and antagonists include leuprorelin (Lupron), goserelin (Zoladex), degarelix, ozarelix, elagolix (ABT-620), relugolix (TAK-385), EP-100, or KLH-2109. Combinations of GnRH agonist or antagonists or antagonists can also be included in the core. Pharmaceutically acceptable salts, esters, or prodrugs of GnRH agonist or antagonists (e.g., Goserelin acetate) can also be utilized herein.

In embodiments, the GnRH agonist or antagonist includes goserelin acetate. Goserelin acetate is an acetate salt of [D-Ser(Bu^{t})6,Azgly¹⁰] having the following chemical structure: pyro-Glu-His-Trp-Ser-Tyr-D-Ser(Bu^{t})-Leu-Arg-Pro-Azgly-NH₂ acetate [C₅₉H₈₄N₁₈O₁₄⁻(C₂H₄O₂)ₓ where x=1 to 2.40]. Goserelin acetate has a molecular weight of 1269 Daltons (free base). Goserelin acetate is a synthetic decapeptide analogue of GnRH and acts as an inhibitor of pituitary gonadotropin secretion when administered.

In certain other embodiments, the GnRH agonist or antagonist can include molecules that are analogues of GnRH or act to inhibit gonadotropin secretion. For instance, in certain embodiments the GnRH agonist or antagonist can include small molecules or peptide fragments.

### III. Membrane Layer(s)

As indicated above, the implantable device can optionally include one or more membrane layers (e.g., a first membrane layer) that is positioned adjacent to an outer surface of a core. Additional membrane layers (e.g., a second membrane layer, a third membrane layer, etc.) may be layered on the core as desired. The number of membrane layers may vary depending on the particular configuration of the device, the nature of the therapeutic agent, and the desired release profile.

Regardless of the particular configuration employed, the membrane polymer matrix contains at least one ethylene vinyl acetate copolymer, such as described in more detail above. The vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The melting temperature of the ethylene vinyl acetate copolymer may also range from about 40° C. to about 140° C., in some embodiments from about 50° C. to about 125° C., and in some embodiments, from about 60° C. to about 120° C., as determined in accordance with ASTM D3418-15. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA®4030AC); Dow under the designation ELVAX® (e.g., ELVAX®40W); and Arkema under the designation EVATANE® (e.g., EVATANE 40-55). In embodiments, the ethylene vinyl acetate copolymer in the membrane polymer matrix is from about 20 wt. % to about 90 wt. %, such as from about 30 wt. % to about 80 wt. %, such as from about 40 wt. % to about 70 wt. %.

In certain cases, ethylene vinyl acetate copolymer(s) constitute the entire polymer content of the membrane polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix. The membrane polymer matrix typically constitutes from about 50 wt. % to 99 wt. %, in some embodiments, from about 55 wt. % to about 98 wt. %, in some embodiments from about 60 wt. % to about 96 wt. %, and in some embodiments, from about 70 wt. % to about 95 wt. % of a membrane layer.

To help further control the release rate from the implantable medical device, a hydrophilic compound may also be incorporated into the membrane layer(s) that is soluble and/or swellable in water. When employed, the weight ratio of the ethylene vinyl acetate copolymer(s) the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the membrane layer(s) can include a plurality of water-soluble particles distributed within a membrane polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of a membrane layer.

When employing multiple membrane layers, it is typically desired that each membrane layer contains a polymer matrix including an ethylene vinyl acetate copolymer. Additionally, each of the membrane layers can include a plurality of water-soluble particles distributed within a membrane polymer matrix that includes an ethylene vinyl acetate copolymer. For example, a first membrane layer may contain first water-soluble particles distributed within a first membrane polymer matrix and a second membrane layer may contain second water-soluble particles distributed within a second membrane polymer matrix. In such embodiments, the first and second polymer matrices may each contain an ethylene vinyl acetate copolymer. The water-soluble particles and ethylene vinyl acetate copolymer(s) within one membrane layer may be the same or different than those employed in another membrane layer. In one embodiment, for instance, both the first and second membrane polymer matrices employ the same ethylene vinyl acetate copolymer(s) and the water-soluble particles within each layer have the same particle size and/or are formed from the same material. Likewise, the ethylene vinyl acetate copolymer(s) used in the membrane layer(s) may also be the same or different the hydrophobic polymer(s) employed in the core. In one embodiment, for instance, both the core and the membrane layer(s) employ the same ethylene vinyl acetate copolymer. In yet other embodiments, the membrane layer(s) may employ an ethylene vinyl acetate copolymer that has a lower melt flow index than a hydrophobic polymer employed in the core. Among other things, this can further help control the release of the therapeutic agent from the device. For example, the ratio of the melt flow index of a hydrophobic polymer employed in the core to the melt flow index of an ethylene vinyl acetate copolymer employed in the membrane layer(s) may be from about 1 to about 20, in some embodiments about 2 to about 15, and in some embodiments, from about 4 to about 12.

If desired, membrane layer(s) used in the device may optionally contain a therapeutic agent, such as described above, which is also dispersed within the membrane polymer matrix. The therapeutic agent in the membrane layer(s) may be the same or different than the therapeutic agent employed in the core. When such a therapeutic agent is employed in a membrane layer, the membrane layer generally contains the therapeutic agent in an amount such that the ratio of the concentration (wt. %) of the therapeutic agent in the core to the concentration (wt. %) of the therapeutic agent in the membrane layer is greater than 1, in some embodiments about 1.5 or more, and in some embodiments, from about 1.8 to about 4. When employed, therapeutic agents typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of therapeutic agents prior to release from the core. When multiple membrane layers are employed, each membrane layer may generally contain the therapeutic agent in an amount such that the ratio of the weight percentage of the therapeutic agent in the core to the weight percentage of the therapeutic agent in the membrane layer is greater than 1, in some embodiments about 1.5 or more, and in some embodiments, from about 1.8 to about 4.

The membrane layer(s) may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

One or more nonionic, anionic, and/or amphoteric surfactants may also be employed to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of the core or membrane layers. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN®80, or polyethylene (20) sorbitan monooleate).

The membrane layer(s) may be formed using the same or a different technique than used to form the core, such as by hot-melt extrusion, compression molding (e.g., vacuum compression molding), injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc. Compression molding (e.g., vacuum compression molding) may also be employed to form the implantable device. As described above, the core and membrane layer(s) may be each individually formed by heating and compressing the respective polymer compression into the desired shape while under vacuum. Once formed, the core and membrane layer(s) may be stacked together to form a multi-layer precursor and thereafter and compression molded in the manner as described above to form the resulting implantable device.

As indicated above, the implantable device can include at least one membrane layer that is positioned adjacent to an outer surface of a core. The number of membrane layers may vary depending on the particular configuration of the device, the nature of the therapeutic agent, and the desired release profile. For example, the device may contain only one membrane layer. The thickness of the membrane layer depends on the desired release for the therapeutic agent. However, in embodiments the membrane layer has a membrane thickness of from about 100 microns to about 500 microns, such as from about 200 microns to about 300 microns, such as from about 150 microns to about 350 microns.

Regardless of the particular configuration employed, the membrane layer(s) generally contain a membrane polymer matrix that contains a hydrophobic polymer, such as described above. The polymer matrix typically constitutes from about 30 wt. % to 100 wt. %, in some embodiments, from about 40 wt. % to about 99 wt. %, and in some embodiments, from about 50 wt. % to about 90 wt. % of a membrane layer. When employing multiple membrane layers, it is typically desired that each membrane layer contains a polymer matrix that includes such a hydrophobic polymer. For example, a first membrane layer may contain a first polymer matrix and a second membrane layer may contain a second polymer matrix. In such embodiments, the first and second polymer matrices each contain a hydrophobic polymer, which may be the same or different. Likewise, the hydrophobic polymer used in the membrane layer may also be the same or different the hydrophobic polymer employed in the core. In one embodiment, for instance, both the core and the membrane layer(s) employ the same hydrophobic polymer (e.g., α-olefin copolymer). In yet other embodiments, the membrane layer(s) may employ a hydrophobic polymer (e.g., α-olefin copolymer) that has a lower melt flow index than a polymer employed in the core. Among other things, this can further help control the release of the therapeutic agent from the device. For example, the ratio of the melt flow index of a hydrophobic polymer employed in the core to the melt flow index of a hydrophobic polymer employed in the membrane layer(s) may be from about 1 to about 20, in some embodiments about 2 to about 15, and in some embodiments, from about 4 to about 12. The melt flow index of the hydrophobic polymer in the membrane layer(s) may, for example, range from about 1 to about 80 g/10 min, in some embodiments from about 2 to about 70 g/10 min, and in some embodiments, from about 5 to about 60 g/10 min, as determined in accordance with ASTM D1238-13 at a temperature of 190° C. and a load of 2.16 kilograms. Examples of suitable ethylene vinyl acetate copolymers that may be employed include those available from Celanese under the designation ATEVA® (e.g., ATEVA®4030AC or 2861A).

As indicated above, the membrane layer(s) used in the device may optionally contain a therapeutic agent (e.g., a GnRH agonist or antagonist), such as described above, which is dispersed within the polymer matrix. The therapeutic agent in the membrane layer(s) may be the same or different than the therapeutic agent employed in the core. Regardless, when such a therapeutic agent is employed in a membrane layer, the membrane layer generally contains the therapeutic agent in an amount such that the ratio of the concentration (wt. %) of the therapeutic agent in the core to the concentration (wt. %) of the therapeutic agent in the membrane layer is greater than 1, in some embodiments about 1.5 or more, and in some embodiments, from about 1.8 to about 4. When employed, therapeutic agents typically constitute only about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of such therapeutic agents prior to release from the core. When multiple membrane layers are employed, each membrane layer may generally contain the therapeutic agent in an amount such that the ratio of the weight percentage of the therapeutic agent in the core to the weight percentage of the therapeutic agent in the membrane layer is greater than 1, in some embodiments about 1.5 or more, and in some embodiments, from about 1.8 to about 4.

The membrane layer(s) and/or the core may also optionally contain one or more excipients as described above, such as radiocontrast agents, hydrophilic compounds, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

To help further control the release rate from the implantable device, for example, a hydrophilic compound may also be incorporated into the polymer matrix of the membrane layer(s) that is soluble and/or swellable in water. When employed, the weight ratio of the hydrophobic polymers to the hydrophilic compounds within the membrane polymer matrix may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 50 wt. %, in some embodiments from about 2 wt. % to about 40 wt. %, and in some embodiments, from about 5 wt. % to about 30 wt. % of the membrane polymer matrix, and in some embodiments 30 wt. % to about 50 wt. % of the membrane polymer matrix, and in some embodiments 40 wt. % to about 50 wt. % of the membrane polymer matrix, while hydrophobic polymers typically constitute from about 50 wt. % to about 99 wt. %, in some embodiments from about 60 wt. % to about 98 wt. %, and in some embodiments, from about 70 wt. % to about 95 wt. % of the membrane polymer matrix. In such embodiments, hydrophilic compounds may likewise constitute from about 1 wt. % to about 50 wt. %, in some embodiments from about 2 wt. % to about 40 wt. %, and in some embodiments, from about 5 wt. % to about 30 wt. %, and in some embodiments 30 wt. % to about 50 wt. %, and in some embodiments from about 40 wt. % to about 50 wt. % of a membrane layer. Suitable hydrophilic compounds may include, for instance, polymers, non-polymeric materials (e.g., glycerin, sugars, salts, peptides, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc. The one or more hydrophilic compounds can include particles having an average particle size of from about 75 microns to about 200 microns, such as from about 100 microns to about 200 microns, such as from about 100 microns to about 150 microns.

The membrane layer(s) may be formed using the same or a different technique than used to form the core, such as by hot-melt extrusion, injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc.

### IV. Use of Device

The implantable device of the present invention may be used in a variety of different ways to treat prohibit and/or treat a condition, disease, or cosmetic state in a patient. The device may be implanted subcutaneously, orally, rectally, mucosally, etc., using standard techniques. The delivery route may be intrapulmonary, gastroenteral, subcutaneous, intramuscular, or for introduction into the central nervous system, intraperitoneum or for intraorgan delivery. The term “implantable device” as used herein, is intended to cover a variety of implantable or insertable devices and associated methods of use. For example, the implantable device can be inserted subcutaneously using standard techniques. In such embodiments, the delivery route may be subcutaneous.

As noted above, the implantable device may be particularly suitable for delivering a GnRH agonist or antagonist for the treatment of cancer (e.g., breast cancer, prostate cancer, etc.). The device may also be employed together with other therapies for the treatment of cancer, including aromatase inhibitors, cytotoxic agents (e.g., chemotherapeutic agents), etc. In such embodiments, these additional therapeutic agents can be administered to the patient in a variety of dosage forms, including, oral dosage forms, intravenous dosage forms, subcutaneous dosage forms, including depot injections, hydrogel injections, intramuscular injections, etc., or intravaginal dosage forms. Additional therapeutic agents can be administered via any suitable route and can be used in combination with the implantable device disclosed herein. The implantable device may also be particularly suitable for treating endometriosis and/or endometrial thinning.

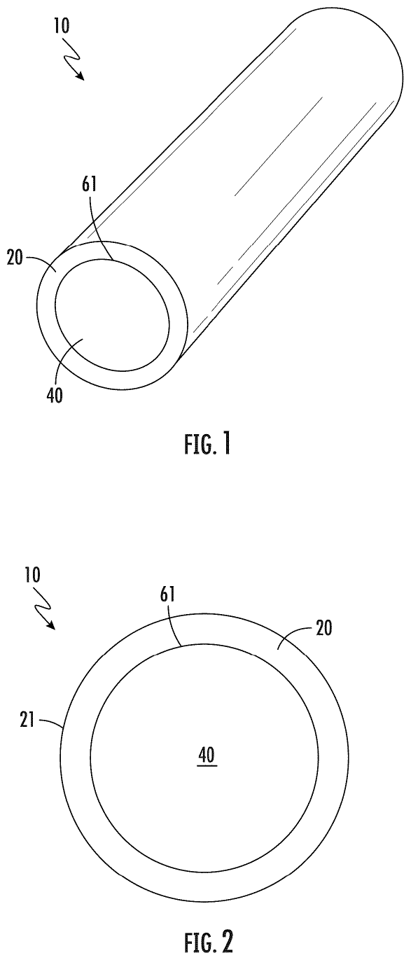

Referring to FIGS. 1-2, for example, one embodiment of an implantable device 10 is shown that contains a core 40 having a generally circular cross-sectional shape and is elongated so that the resulting device is generally cylindrical in nature. The core 40 defines an outer circumferential surface 61 about which a membrane layer 20 is circumferentially disposed. Similar to the core 40, the membrane layer 20 also has a generally circular cross-sectional shape and is elongated so that it covers the entire length of the core 40. During use of the implantable device 10, a therapeutic agent is capable of being released from the core 40 and through the membrane layer 20 so that it exits from an external surface 21 of the device. While an implantable device 10 having a core 40 and membrane layer 20 is depicted, it should be appreciated that the membrane layer 20 is optional and the implantable device 10 can be a monolithic implant containing only the core 40.

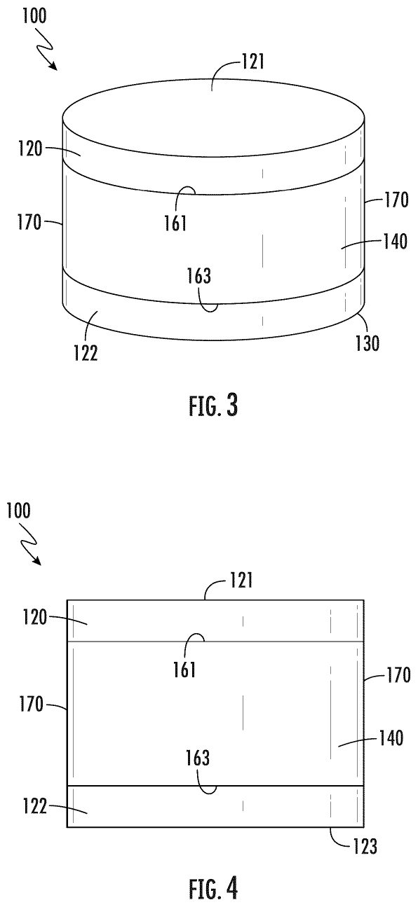

Of course, in other embodiments, the device may contain multiple membrane layers. In the device of FIGS. 1-2, for example, one or more additional membrane layers (not shown) may be disposed over the membrane layer 20 to help further control release of the therapeutic agent. In other embodiments, the device may be configured so that the core is positioned or sandwiched between separate membrane layers. Referring to FIGS. 3-4, for example, one embodiment of an implantable device 100 is shown that contains a core 140 having a generally circular cross-sectional shape and is elongated so that the resulting device is generally disc-shaped in nature. The core 140 defines an upper outer surface 161 on which is positioned a first membrane layer 120 and a lower outer surface 163 on which is positioned a second membrane layer 122. Similar to the core 140, the first membrane layer 120 and the second membrane layer 122 also have a generally circular cross-sectional shape that generally covers the core 140. If desired, edges of the membrane layers 120 and 122 may also extend beyond the periphery of the core 140 so that they can be sealed together to cover any exposed areas of an external circumferential surface 170 of the core 140. During use of the implantable device 100, a therapeutic agent is capable of being released from the core 140 and through the first membrane layer 120 and second membrane layer 122 so that it exits from external surfaces 121 and 123 of the device. Of course, if desired, one or more additional membrane layers (not shown) may also be disposed over the first membrane layer 120 and/or second membrane layer 122 to help further control release of the therapeutic agent.

The implantable device may have a variety of different geometric shapes, such as cylindrical (rod), disc, ring, doughnut, helical, elliptical, triangular, ovular, etc. In one embodiment, for example, the device may have a generally circular cross-sectional shape so that the overall structure is in the form of a cylinder (rod) or disc. In such embodiments, the device will typically have a diameter of from about 0.5 to about 50 millimeters, in some embodiments about 1 to about 40 millimeters, and in some embodiments, from about 5 to about 30 millimeters. In some embodiments, the device has a diameter of about 2 millimeters to about 4 millimeters. The length of the device may vary, but is typically in the range of about 1 to about 60 millimeters, in some embodiments from about 20 to about 40 millimeters. Cylindrical devices may, for instance, have a length of about 5 to about 50 millimeters, while disc-shaped devices may have a length of from about 0.5 to about 5 millimeters. Certain disc-shaped devices may have a thickness ranging from about 1 mm to about 5 mm, such as from about 2 mm to about 4 mm, such as about 3 mm, and a disc diameter ranging from about 0.5 cm to about 5 cm, such as from about 1 cm to about 4 cm, such as from about 2 cm to about 3 cm.

Through selective control over the particular nature of the device and the manner in which it is formed, the resulting device can be effective for sustained release of one or more GnRH agonist or antagonists over a prolonged period of time. For example, the implantable device can release the therapeutic agent(s) for a time period of about 5 days or more, in some embodiments about 10 days or more, in some embodiments from about 21 days or more, and in some embodiments, from about 25 days to about 50 days (e.g., about 30 days). In certain embodiments, the device can release the therapeutic agent(s) for a time period of about 1 month or more, such as about 3 months or more, such as about 6 months or more, such as about 12 month or more, and in some embodiments, from about 12 months to about 36 months. Further, the therapeutic agent(s) can be released in a controlled manner (e.g., zero order or near zero order) over the course of the release time period. After a time period of 21 days, for example, the cumulative release ratio of the implantable device may be from about 10% to about 70%, in some embodiments from about 30% to about 65%, and in some embodiments, from about 40% to about 60%. Likewise, after a time period of 30 days, the cumulative release ratio of the implantable device may still be from about 40% to about 85%, in some embodiments from about 50% to about 80%, and in some embodiments, from about 60% to about 80%. The “cumulative release ratio” may be determined by dividing the amount of the therapeutic agent released at a particulate time interval by the total amount of therapeutic agent initially present, and then multiplying this number by 100.

The implantable device can exhibit a reduced initial burst effect when introduced into the tissue of a patient. For instance, the implantable device can exhibit a cumulative release of less than about 5%, such as less than about 10%, such as less than about 12.5%, such as less than about 15% within a time period of about 3 days.

Of course, the actual dosage level of the GnRH agonist or antagonist delivered will vary depending on the particular GnRH agonist or antagonist employed and the time period for which it is intended to be released. The dosage level is generally high enough to provide a therapeutically effective amount of the GnRH agonist or antagonist to render a desired therapeutic outcome, i.e., a level or amount effective to reduce or alleviate symptoms of the condition for which it is administered. The exact amount necessary will vary, depending on the subject being treated, the age and general condition of the subject to which the GnRH agonist or antagonist is to be delivered, the capacity of the subject's immune system, the degree of effect desired, the severity of the condition being treated, the particular GnRH agonist or antagonist selected and mode of administration of the composition, among other factors. For example, an effective amount will typically range from about 0.01 mg to about 2 mg per day, such as from about 0.1 mg to about 1 mg per day, such as from about 0.1 mg to about 0.5 mg per day of the GnRH agonist or antagonist. In certain embodiments, an effective amount ranges from about 0.1 mg to about 0.5 mg per day. In embodiments, the device provides a therapeutic dose of between about 0.1 mg to about 0.5 mg of the therapeutic agent per day for a time period of at least 60 days. In other embodiments, the device provides a therapeutic dose of between about 0.1 mg to about 0.5 mg of the therapeutic agent per day for a time period of at least about 90 days. In other embodiments, one or more GnRH agonists or antagonists are released from the device in an amount sufficient to deliver from about 0.001 mg of GnRH agonist or antagonist to about 4 mg of GnRH agonist or antagonist per day.

The amount of GnRH agonist or antagonist loaded into the device can vary. For example, for implantable devices configured to be inserted and retained for periods of time ranging from about one month but fewer than 12 months, the core can be loaded with 100 mg to about 400 mg, such as about 150 mg to about 350 mg, such as about 200 mg to about 300 mg of one or more GnRH agonist or antagonist. In embodiments, the core is loaded with from about 3 mg to about 365 mg of one or more GnRH agonist or antagonist. Additionally, the amount of GnRH agonist or antagonist loaded into the core can be modified (e.g., increased and/or decreased) depending on the amount of implantation time desired or route of implantation. Additionally, the amount of GnRH agonist or antagonist loaded into the core can be modified based on the use of additional therapeutic agents in addition to the GnRH agonist or antagonists. For example, the amount of GnRH agonist or antagonist loaded into the core can be increased when the implant includes or is co-administered with one or more therapeutic agents known to increase blood glucose levels.

If desired, the device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

### Examples 1-3

Rod-shaped implants containing goserelin acetate (GA) and ethylene vinyl acetate copolymer (EVA) were produced via hot melt extrusion. The implants contained 40 wt. % Ateva® 4030AC and 60 wt. % GA. EVA was cryogenically ground to a powder and then blended by hand with GA powder. The resulting powder blend was fed into an 11 mm twin-screw extruder. Extrusion was accomplished using a screw speed of 50 rpm with barrel temperatures set to achieve a nominal melt temperature of 65° C. The extrudate was approximately 3 mm in diameter, and was cut to 1 cm length rods.

For Example 1, the extrudate was cut into small pieces. The extrudate pieces underwent secondary processing by using the method of vacuum compression molding (MeltPrep VCM). VCM involved subjecting the small pieces to a temperature of 65° C. for a duration of 15 minutes, fusing them together into a continuous solid rod, after which they were cooled using air for 3 minutes. The diameter of the resulting rod was roughly 2 mm, while its length was approximately 10 mm.

For Examples 2-3 two mannitol samples with very different particle sizes were obtained and membrane layer were made according to the following. For Example 2, a polydisperse mannitol sample (Sigma-Aldrich) was fractionated using a sieve shaker system to prepare a fraction of mannitol with particle sizes ranging between 125-200 μm; this sample is referred to as Mannitol (125 μm). For Example 3, a second mannitol sample with a nominal particle size of 25 μm (Roquette) was used as received; we refer to this sample as Mannitol (25 μm).

Mannitol/EVA compounds used to produce implant membranes were prepared via hot melt extrusion following the method described hereinabove. Two compositions were produced. The compound used to produce the membrane layer of Example 2 consisted of 55% VitalDose® 3240.X01 (40% vinyl acetate, melt index=55 g/10 min) and 45% Mannitol (125 μm). The compound used to produce the membrane layer of implant Example 3 consisted of 65% Ateva® 4030AC (40% vinyl acetate, melt index=55 g/10 min) and 35% Mannitol (25 μm).

Vacuum compression molding was used to produce two-layer core-membrane implants. Cores identical to Example 1 were first prepared. Example 2 consisted of this core surrounded by a 250 μm layer of 55% VitalDose® 3240.X01/45% Mannitol (125 μm). Example 3 consisted of this core surrounded by a 250 μm layer of 65% Ateva® 4030ACG/35% Mannitol (25 μm).

The fabrication of Examples 2 and 3 consisted of a two-step process. In the first step, membranes were molded using vacuum compression molding (MeltPrep VCM) at a temperature of 65° C. for a duration of 10 minutes, and thereafter cooling them with air for 3 minutes. In the second step, membranes were attached to cores using vacuum compression molding at a temperature of 65° C. for a duration of 15 minutes, followed by a cooling process with air for 3 minutes.

In vitro drug elution testing was conducted by immersing a single implant in 25 mL of phosphate buffered saline solution with a pH of 7.4 containing 0.02% (w/v) sodium azide. Media and implant were placed in an incubator at 37° C. and were gently agitated on a 100 rpm oscillating table. On a periodic basis, elution media was replaced with fresh buffer, and the concentration of GA in the media was measured via HPLC. Through quantification of solution concentration, it was possible to determine the total amount of drug released since the previous sampling interval; these data were used to build a cumulative release curve.

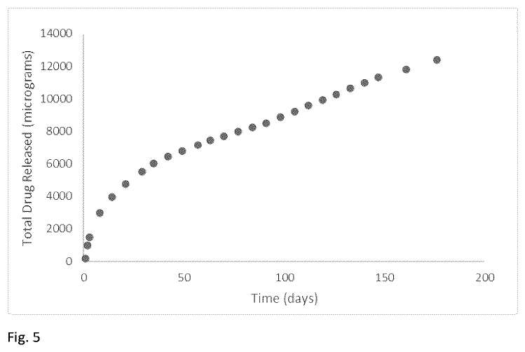

The cumulative drug release as a function of time is shown in FIG. 5. FIG. 5 illustrates the cumulative release (mg) per day of goserelin acetate for Examples 1-3 and Table 1 below provides the data points for goserelin acetate release as shown in FIG. 5.

TABLE 1

| | Example 1 | Example 2 | Example 3 |
|---|---|---|---|
| | cumulative | cumulative | cumulative |
| Time (days) | release (mg) | release (mg) | release (mg) |
| 1 | 4.81 | 0.52 | 0.03 |
| 2 | 6.35 | 0.88 | 0.03 |
| 3 | 7.67 | 1.26 | 0.03 |
| 7 | 10.08 | 2.29 | 0.03 |
| 14 | 12.89 | 3.66 | 0.03 |
| 17 | 13.76 | 4.26 | 0.07 |
| 24 | 15.27 | 5.57 | 0.16 |
| 28 | 15.93 | 6.39 | 0.27 |
| 35 | 16.53 | 7.35 | 0.38 |
| 49 | 16.75 | 8.96 | 0.60 |
| 63 | 16.84 | 10.23 | 0.76 |
| 77 | 16.91 | 11.23 | 0.90 |
| 92 | 16.95 | 12.00 | 0.99 |

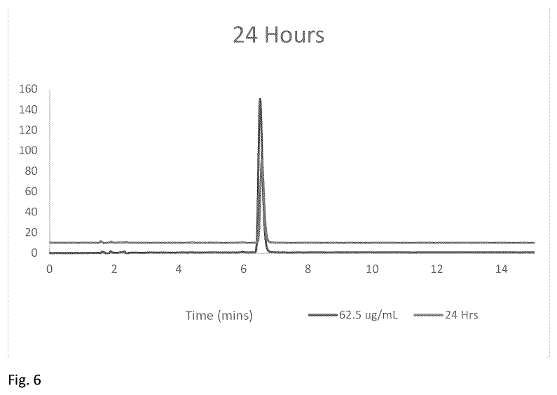

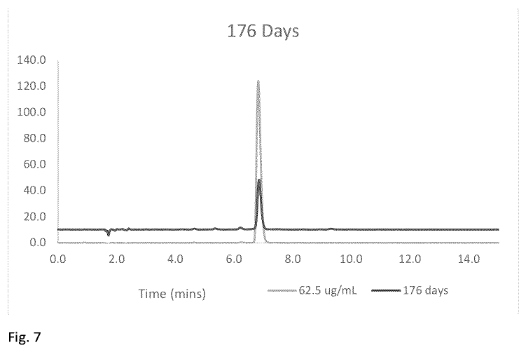

To verify that goserelin acetate was not altered during implant manufacture, the HPLC chromatograms were compared with a goserelin acetate solution. FIG. 6 illustrates HPLC chromatograms taken at Day 1 for Examples 1-3 and FIG. 7 illustrates HPLC chromatograms taken at Day 92 for Examples 1-3.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. An implantable device for delivery of a therapeutic agent, the device comprising:
a core comprising a core polymer matrix within which is dispersed a therapeutic agent comprising one or more gonadotropin-releasing hormone (GnRH) agonist or antagonist, the core polymer matrix includes a first ethylene vinyl acetate copolymer having a melting temperature of from about 20° C. to about 100° C. as determined in accordance with ASTM D3418-15 and/or a melt flow index of from about 0.2 to about 100 grams per 10 minutes as determined in accordance with ASTM D1238-13 at a temperature of 190° C. and a load of 2.16 kilograms.

2. The implantable device of claim 1, wherein the ethylene vinyl acetate copolymer has a vinyl acetate monomer content of from about 10 wt. % to about 50 wt. % of the copolymer.

3. The implantable device of claim 1, wherein the gonadotropin-releasing hormone agonist or antagonist comprises leuprorelin, goserelin, degarelix, ozarelix, elagolix, EP-100, KLH-2109, or combinations thereof.

4. The implantable device of claim 1, wherein therapeutic agent constitutes from about 30 wt. % to about 75 wt. % of the core and the core polymer matrix constitutes from about 20 wt. % to about 80 wt. % of the core.

5. The implantable device of claim 1, comprising a membrane layer disposed on an outer surface of the core, the membrane layer including a membrane polymer matrix containing a second ethylene vinyl acetate copolymer.

6. The implantable device of claim 5, wherein the membrane polymer matrix comprises one or more hydrophilic compounds to control release of the therapeutic agent from the device.

7. The implantable device of claim 6, wherein the one or more hydrophilic compounds constitute from about 30 wt. % to about 50 wt. % of the membrane polymer matrix.

8. The implantable device of claim 7, wherein the one or more hydrophilic compounds constitute from about 40 wt. % to about 50 wt. % of the membrane polymer matrix.

9. The implantable device of claim 6, wherein the one or more hydrophilic compounds comprise particles having an average particle size of from about 75 microns to 200 microns.

10. The implantable device of claim 6, wherein the one or more hydrophilic compounds comprise particles having an average particle size of from about 10 microns to about 40 microns.

11. The implantable device of claim 5, wherein the membrane layer has a membrane thickness of about 200 microns to about 300 microns.

12. The implantable device of claim 1, wherein the device provides a therapeutic dose of between about 0.1 mg to about 0.5 mg of the therapeutic agent per day for a time period of at least 60 days.

13. The implantable device of claim 1, wherein the device provides a therapeutic dose of between about 0.1 mg to about 0.5 mg of the therapeutic agent per day for a time period of at least 90 days.

14. The implantable device of claim 1, wherein the device exhibits a cumulative release of less than about 12.5% within a time period of about 3 days.

15. The implantable device of claim 1, wherein the core is loaded with from about 3 mg to about 365 mg of one or more GnRH agonist or antagonists.

16. The implantable device of claim 1, wherein the one or more GnRH agonists or antagonists are released from the device in an amount sufficient to deliver from about 0.001 mg of GnRH agonist or antagonist to about 4 mg of GnRH agonist or antagonist per day.

17. The implantable device of claim 1, wherein the core contains a radiocontrast agent.

18. A method for prohibiting and/or treating a condition, disease, and/or cosmetic state of a patient, the method comprising subcutaneously implanting the implantable device of claim 1 in the patient.

19. A method of manufacturing an implantable device, the method comprising:
hot melt extruding a core polymer matrix comprising a first ethylene vinyl acetate copolymer and a therapeutic agent, the therapeutic agent comprising one or more gonadotropin-releasing hormone (GnRH) agonist or antagonist, to form a first core material;
hot melt extruding a membrane polymer matrix comprising one or more hydrophilic compounds with a second ethylene vinyl acetate copolymer forming a first membrane material; and
coextruding the first core material and the first membrane material to form the implantable medical device.

20. The method of claim 19, wherein the one or more hydrophilic compounds constitute from about 30 wt. % to about 50 wt. % of the membrane polymer matrix.
